(19) United States
(12) Patent Application Publication
(10) Pub. No.: US 2014/0005266 A1
(43) Pub. Date: Jan. 2, 2014

(54) TOPICAL FORMULATIONS FOR ADMINISTRATION OF OMEGA-3 FATTY ACIDS

(71) Applicants: Nirmal Mulye, Kendall Park, NJ (US); David Assoulin, Deal, NJ (US)

(72) Inventors: Nirmal Mulye, Kendall Park, NJ (US); David Assoulin, Deal, NJ (US)

(73) Assignee: NOSTRUM LABORATORIES, INC., New Brunswick, NJ (US)

(21) Appl. No.: 13/991,020

(22) PCT Filed: Nov. 30, 2011
(86) PCT No.: PCT/US2011/062559

§ 371 Date: Sep. 19, 2013

Related U.S. Application Data
(60) Provisional application No. 61/418,705, filed on Dec. 1, 2010.
(60) Provisional application No. 61/422,264, filed on Dec. 13, 2010.

(51) Int. Cl. A61K 31/202; A61K 9/00; A61Q 19/08; A61K 8/36

(52) U.S. Cl. CPC A61K 31/202; A61K 8/361; A61K 9/0014; A61Q 19/08
USPC 514/560

(57) ABSTRACT

A pharmaceutical formulation is provided, for application directly to human skin comprising about 5% to 50% by weight of omega-3 fatty acids derived from fish oil or a vegetable source. The formulation may be an ointment or water-based emulsion. The formulation may be used to treat skin diseases, cardiovascular conditions, and is also useful as a general skin care or beauty product that reverses the effect of aging on the skin.

### CROSS REFERENCE TO RELATED APPLICATION APPLICATIONS

This application claims priority to U.S. Patent Application Nos. 61/418,705, filed Dec. 1, 2010, and 61/422,264, filed Dec. 13, 2010, the contents of which are incorporated by reference.

### FIELD OF THE INVENTION

This invention pertains to topical skin preparations, such as creams or ointments for application to the skin, containing a high omega-3 fatty acid content, such as derived from a fish oil.

### BACKGROUND

Omega-3 fatty acids are a family of polyunsaturated fatty acids that have a final carbon-carbon double bond in the n-3 position; that is, the third bond from the methyl end of the fatty acid. These fatty acids are nutritionally important in humans. Nutritionally important omega-3 fatty acids include α-linolenic acid (ALA), eicosapentaenoic acid (EPA), and docosahexaenoic acid (DHA), all of which are polyunsaturated. These fatty acids have an even number of carbon atoms and multiple cis-double bonds.

The human body cannot synthesize omega-3 fatty acids de novo, but it can form long chain 20-carbon unsaturated omega-3 fatty acids (like EPA) and 22-carbon unsaturated omega-3 fatty acids (like DHA) from the “short chain” eighteen-carbon omega-3 fatty acid α-linolenic acid. The short chain omega-3 fatty acids are converted to long chain forms (EPA, DHA) with an efficiency of approximately 5% in men, and at a greater percentage in women. These conversions occur competitively with omega-6 fatty acids, which are essential closely related chemical analogues that are derived from linoleic acid. Both the omega-3 α-linolenic acid and omega-6 linoleic acid must be obtained from food. Synthesis of the longer omega-3 fatty acids from linolenic acid within the body is competitively slowed by the omega-6 analogues. Thus accumulation of long-chain omega-3 fatty acids in tissues is more effective when they are obtained directly from food or when competing amounts of omega-6 analogs do not greatly exceed the amounts of omega-3 fatty acids.

Omega-3 fatty acids can be obtained from a number of sources, including vegetable, animal, and fish oils. Many experts believe that fish oils provide the most beneficial blend of fatty acids. It is believed that fish do not actually produce fatty acid in their bodies, but rather accumulate the oils from either consuming omega-3-containing algae or plankton, or from consuming prey fish that consume omega-3 oils.

A number of plant species are also sources of omega-3 fatty acids, particularly in the seed oil, including perilla seeds (Linnaean name Perilla frutescens); chia seeds (Salvia hispanica); flax seed (Linum usitatissimum); lingon berry seeds (Vaccinium vitis-idaea); and rapeseed (Brassica napus).

Current thinking is that omega-3 fatty acids have important health benefits and are useful for treating certain diseases and medical conditions. Moreover, arachidonic acid, an omega-6 fatty acid, has been implicated in undesirable enhanced inflammatory responses in the arachidonic acid cascade. Thus, because of the competitive conversion of shorter chain fatty acids, low omega-3 fatty acid dietary intake is implicated in the so-called “diseases of western civilization” (e.g., Alzheimer's Disease, cancer, asthma, Crohn's disease), that involve inflammatory responses. By contrast, dietary omega-3 fatty acid supplements have been reported to reduce risk factors for heart disease, including high cholesterol and high blood pressure, lower levels of triglycerides, prevent and treat atherosclerosis, lower triglycerides and apoproteins, and raise HDL. Other conditions reportedly improved by omega-3 fatty acid dietary intake include reducing symptoms of lupus, treating psychiatric disorders, preventing and treating several forms of cancer, preventing macular degeneration, and treating arthritis.

In particular, omega-3 fatty acid dietary supplementation has been reported to treat skin conditions, including dry skin conditions and psoriasis. In most prior art investigations of treatments of skin conditions with omega-3 fatty acids, the fatty acids were administered orally.

Direct application of omega-3 fatty acids to the skin has not been fully investigated. For example, Henneicke-Von Zepelin et al. (Br. J. Dermatology, (1993) 129, 713; doi: 10.1111/j.1365-2133.1993.tb03338.x) investigated the use of omega-3 fatty acid esters as topical ointments for psoriasis. The authors concluded that the ointments were ineffective. Similarly, Dewsbury et al. (Br. J. Dermatology, (1989) 120, 581; doi 10.1111/j.1365-2133.1989.tb01335.x) reported on an ointment in an oily base (unguentum) using fish oil from commercially available capsules. The ointment of Dewsbury et al. is of uncertain content; neither the fish oil contents nor the composition of the oily base are disclosed. As noted below, there is poor absorption into the skin from many ointments, which is a factor addressed in this invention. WO2011/056327 discloses antibacterial activity in oral use of topical preparations containing polyunsaturated fatty acids, such as omega-3, omega-6, and omega-9 fatty acids (i.e., topically applied to the teeth and gums). None of these references disclose the simple and expedient of this invention of very high omega-3 fatty acid topical formulations that absorb into the skin.

### SUMMARY OF THE INVENTION

This invention provides transdermal formulation containing 5% to about 50% by weight of omega-3 fatty acids, for direct application to the skin, useful for treating medical conditions responsive to omega-3 fatty acid therapy, including psoriasis, dermatitis, lowering triglyerides or LDL cholesterol, raising HDL cholesterol, cancers, and as beauty preparations. The skin preparation may be in the form of a cream, ointment, or balm, such as a deodorant stick or lip balm.

Additionally, a method of administering omega-3 fatty acids to a patient is provided, wherein omega-3 fatty acids are formulated into an ointment or cream for application directly to the skin of the patient. The omega-3 fatty acids are absorbed directly in to the skin for treatment of dermal diseases, or are absorbed into the blood for treatment and prevention of internal diseases, including cardiovascular conditions, neurological conditions, bone and joint conditions, and certain cancers. Additionally, the method is of value as a beauty treatment, to restore elasticity and a healthy appearance to skin.

The formulation and method may be formulated as an ointment or an emulsion (cream) for application directly to the skin.

### DETAILED DESCRIPTION

This invention provides a pharmaceutical formulation for application directly to human skin comprising about 5% to 50% by weight of omega-3 fatty acids. The inventive transdermal formulations are effective for treating skin diseases, including use as beauty treatments, or for treating internal diseases, in which omega-3 fatty acids are known to be of therapeutic value.

Also provided is a method of administering omega-3 fatty acids to a patient, wherein omega-3 fatty acids are formulated into an ointment or cream for application directly to the skin of the patient. The omega-3 fatty acids are absorbed directly in to the skin for treatment of dermal diseases, or are absorbed into the blood for treatment and prevention of internal diseases, including cardiovascular conditions, neurological conditions, bone and joint conditions, and certain cancers.

In an embodiment, the omega-3 fatty acids for the inventive formulations and methods are derived from a marine species, such as a fish oil or crustacean. Examples of fish known to have high levels of omega-3 fatty acids include so-called “oily fish” such as salmon, tuna, swordfish, halibut, tilefish, cod fish (including cod liver oil), anchovies, and sardines. Several crustaceans are also known to have high levels of omega-3 fatty acids, including krill, a crustacean in the Antarctic (the source of krill oil) and the New Zealand green-lipped mussel, (also known as Perna canaliculus). These oils are available in about 15% to over 50% omega-3 fatty acid content by weight.

In another embodiment, the omega-3 fatty acids of the inventive formulations and methods may be derived from a vegetable source, in particular seed oils, including perilla seeds (Linnaean name Perilla frutescens); chia seeds (Salvia hispanica); flax seeds (Linum usitatissimum); lingon berry seeds (Vaccinium vitis-idaea); and rape seeds (Brassica napus), more commonly called canola oil.

The topical formulations of this invention may be formulated as an ointment or a cream. Creams are typically emulsions. Generally, creams are more desirable. The spreadability factor is a major difference between ointment and cream. Ointments are greasier and typically are more viscous and have a thicker consistency than a cream emulsion. Creams have a lighter consistency, and absorb into the skin more quickly than ointments, and work better when coverage of large areas is required. The slower absorption and greasier feel of ointments may make them less desirable for application to large areas of the skin.

The topical formulations may be oil-based, such as an ointment. Such ointments may be based on a pharmaceutical compatible oil, such as mineral oil, petrolatum, or a vegetable oil.

In another embodiment, the formulation may be a water-based emulsion. The emulsion may be a cream. The emulsion may be oil-in-water, or water-in-oil. The oil component may be 15% to 60% by weight of omega-3 fatty acids. In an embodiment, the oil phase of the emulsion may be at least 20% omega-3 fatty acids. The creams as disclosed herein minimize greasiness of the omega-3 fatty acid formulations, which may be desirable. Furthermore, where greasiness is minimized, absorption into the skin is greater, since the greasy sensation is caused by oil that is not absorbed into the skin.

Other ingredients, such as surfactants, penetration enhancers, therapeutic agents, fragrances, and colors may be added to the inventive topical pharmaceutical formulation. In an embodiment, a non-ionic surfactant may be used as an emulsifying agent, such as polysorbate-80 or stearyl alcohol.

The omega-3 fatty acids may be formulated with an antioxidant. This may be particularly important because omega-3 fatty acids are easily oxidized and isomerized, reducing their pharmaceutical effectiveness. For this reason, long term storage of omega-3 fatty acid products can be a problem. For example, butylated hydroxyl anisole (BHA), butylated hydroxyl toluene, or alpha-tocopherol may be added in to the instant formulations.

The instant formulations may also contain an absorption enhancer, such as isopropyl myristate, that improves penetration of the active omega-3 fatty acids into the skin.

The inventive formulations may include at least about 5% by weight of omega-3 fatty acids EPA, DHA, ALA alone or in combination with each other. The concentration can be made higher, for example about 10%, 20%, 40% or 50% omega-3 fatty acids by weight.

In an embodiment, the pharmaceutical formulation may be prepared as a balm, such as a deodorant stick. In another embodiment, the pharmaceutical formulation may be a preparation for application to the lips, such as a lip balm.

The inventive formulations are expected to have several uses, including treatment of skin diseases, as beauty skin care products, and as transdermal delivery vehicles for omega-3 fatty acids.

In an embodiment, the inventive formulations may be used to treat a skin disease, including a dermatitis or a psoriasis condition. By the term “dermatitis” is meant a general class of irritated skin conditions typically involving scaly or excessively dry skin. Eczema is sometimes used interchangeably with “dermatitis,” and includes atopic dermatitis, an immune-mediated inflammation of the skin arising from an interaction between genetic and environmental factors.

The inventive topical formulations are also expected to be useful as transdermal delivery vehicles for omega-3 fatty acids, targeting internal organs. Omega-3 fatty acids have been shown to have beneficial effects in cardiovascular disease, including lowering elevated triglycerides, LDL cholesterol, and VLDL cholesterol, and increasing levels of HDL cholesterol. Omega-3 fatty acids have also shown promise in the prevention and treatment of certain cancers, particularly breast, colon, and prostate cancer. Omega-3 fatty acids have also shown promise in the treatment and prevention of psychiatric disorders, including depression, mood disorders, and dementia conditions. Omega-3 fatty acids have also shown promise in the treatment and prevention of bone and joint diseases, such as rheumatoid arthritis and osteoporosis.

A particular advantage of the inventive topical formulations is that large doses of omega-3 fatty acids can be administered without the need for patients to swallow large amounts of capsules. Besides being inconvenient and simply difficult, oil capsules can cause nausea and stomach upset in many people. The topical dosage forms of the instant invention avoids these problems. Thus, the inventive formulations are expected to be useful for the administration of large doses of omega-3 fatty acids that can be absorbed directly through the skin and into the blood stream for internal use.

The inventive formulations are also expected to be useful as general skin care and beauty preparations. This may include restoring elasticity to the skin of older persons, to provide a more youthful physical appearance. Thus, a method of reversing the effect of aging of the skin is provided, by applying a pharmaceutical formulation for application directly to human skin comprising about 10% to 50% by weight of omega-3 fatty acids.

The instant topical formulations are useful with animals also, including dogs, cats, and livestock, for which administration of omega-3 fatty acids is believed to be beneficial. Thus, the inventive formulations may be applied directly to the skin of a mammal, including pets such as dogs, cats, rabbits, or animals such as livestock.

### Example 1

Salmon Oil Cream. This formula used salmon oil containing 23% omega-3 fatty acids. 4.6 g of the oil contains 1050 mg total omega-3 fatty acids, including EPA, 350 mg, and DHA 450 mg.

Formula:

| Ingredients | Qty (% w/w) |
|---|---|
| Salmon oil (purified) | 35.00 |
| Cetostearyl alcohol | 2.50 |
| Cetomacrogol 1000 | 13.00 |
| Butylated hydroxyl anisole | 0.01 |
| Isopropyl myristate | 10.00 |
| Propylene glycol | 5.00 |
| Polysorbate 80 | 1.00 |
| Methyl paraben | 0.15 |
| Propyl paraben | 0.015 |
| Perfume | 0.60 |
| Purified water | 32.73 |
| Total | 100.00 |

Procedure:

1. Heat salmon oil, cetostearyl alcohol, and cetomacrogol to 70-75° C.
2. Dissolve butylated hydroxyl anisole in isopropyl myristate and maintain at 70-75° C. Add solution from step 2 to step 1 under stirring.
3. Dissolve parabens in propylene glycol and add to the water maintained at temperature of 70-75° C.
4. Emulsification: Add the oil phase slowly to the water phase under high speed stirring.
5. Continue stirring till the cream cools to about 30-35° C.
6. Mix perfume with polysorbate 80 and add to the cream under stirring.

The cream is packed into foil tubes under nitrogen. The total omega-3 fatty acid content of this cream is 8.0%.

A typical application for a skin condition is application to the skin sufficient to cover and wet any lesions, twice per day. A typical application for internal administration of omega-3 fatty acids is 2 to 4 g applied to an inconspicuous body area, such as the inside of the thighs or inside of the forearms, in a divided dose morning and night.

### Example 2

Flax seed oil cream. The same formulation used in Example 1 is prepared, but using flax seed oil instead of salmon oil. The flax seed oil contains 55% alpha-linolenic acid. This cream has 19.25% alpha-linolenic acid by weight.

### Example 3

Salmon Oil-Flax seed oil cream. The same formulation as used in Example is prepared, but using a combination of 20% salmon oil and 15% flax seed oil. This cream has 12.1% omega-3 fatty acids by weight.

### Example 4

A high concentration ointment is prepared as follows with salmon oil.

Formula:

| Ingredients | Qty % w/w |
|---|---|
| Salmon oil | 60 |
| Butylated hydroxyl Anisole | 0.01 |
| Alpha tocopherol | 0.5 |
| Isopropyl myristate | 3.0 |
| White soft paraffin | 28.5 |
| Hard paraffin | 7.0 |
| Perfume | 1.0 |

Procedure:
1. Melt Salmon oil, hard paraffin and part of white soft paraffin and heat to 70-75° C.
2. Dissolve butylated hydroxyl anisole and isopropyl myristate in the remaining white soft paraffin at 70-75° C.
3. Add the product of step 2 to step 1.
4. Cool the ointment under stirring to about 50° C. Add alpha tocopherol and stir.
5. Add perfume at 40° C. under stirring.
6. Cool the ointment to 30-32° C. under stirring.

This ointment is packed into foil tubes under nitrogen. The total omega-3 fatty acid content of this ointment is 13.8%

### Example 5

Flax seed oil ointment. The ointment of Example 4 is prepared, substituting flax seed oil for fish oil. The flax seed oil contains 55% alpha-linolenic acid (ALA). The omega-3 content of this oil (ALA) is 33%.

### Example 6

A 54-year old man, with excema legions on his forearms and elbows applies the cream as prepared in Example 1, twice per day. An immediate improvement is seen, and within a week, the lesions are largely gone. After one week, a maintenance application of once per day is given.

1. A pharmaceutical formulation for topical application directly to human skin consisting essentially of about 5% to about 50% by weight of omega-3 fatty acids, for the treatment of a disease responsive to omega-3 fatty acid therapy.

2. The pharmaceutical formulation of claim 1, wherein the omega-3 fatty acids are derived from a source selected from a marine species.

3. The pharmaceutical formulation of claim 2, wherein the fatty acids are derived from a source selected from a marine species, selected from salmon, tuna, swordfish, halibut, tilefish, cod liver oil, green-lipped muscle, and krill.

4. (canceled)

5. The pharmaceutical formulation of claim 1, wherein the omega-3 fatty acids are derived from a vegetable source.

6. The pharmaceutical formulation of claim 5, wherein the vegetable source of omega-3 fatty acids is selected from Perilla frutescens, Salvia hispanica, Linum usitatissimum, Vaccinium vitis-idaea, and Brassica napus.

7-8. (canceled)

9. The pharmaceutical formulation of claim 1, wherein the formulation is oil-based.

10. The pharmaceutical formulation of claim 1, wherein the preparation is water-based in the form of an emulsion, selected from the group consisting of oil-in-water and water-in-oil.

11. The pharmaceutical formulation of claim 10 a non-ionic surfactant is present.

12. The pharmaceutical formulation of claim 1, which is a deodorant stick.

13. The pharmaceutical formulation of claim 1, for application to the lips.

14. A method of administering omega-3 fatty acids to a patient, comprising applying directly to the skin of said patient a topical formulation consisting essentially of to about 5% to about 50% by weight of omega-3 fatty acids, for the treatment of a disease responsive to omega-3 fatty acid therapy.

15. The method of claim 14, wherein the omega-3 fatty acids are derived from a marine species or a vegetable source.

16. A method of treating a skin disease in a human patient comprising applying directly to the skin of said patient a pharmaceutical formulation consisting essentially of about 10% to 50% by weight of omega-3 fatty acids derived from a marine species.

17. The method of claim 16, wherein the disease is selected from a dermatitis and a psoriasis condition.

18. A method of reversing the effect of aging of the skin in a human patient, comprising applying directly to the skin of said patient a pharmaceutical formulation consisting essentially of about 10% to 50% by weight of omega-3 fatty acids.

19. A method of treating a medical condition in a human patient selected from one or more of elevated triglycerides, elevated LDL cholesterol, elevated VLDL cholesterol, and depressed HDL cholesterol, comprising applying directly to the skin a pharmaceutical formulation consisting essentially of about 5% to 50% by weight of omega-3 fatty acids.

20. A method of treating high triglycerides in a human patient, comprising applying directly to the skin of said patient a pharmaceutical formulation consisting essentially about 10% to 50% by weight of omega-3 fatty acids.

21. (canceled)

22. A pharmaceutical formulation for topical application directly to the skin of a mammal consisting essentially of about 5% to about 50% by weight of omega-3 fatty acids, for the treatment of a disease responsive to omega-3 fatty acid therapy.

23. The pharmaceutical formulation of claim 22 wherein the mammal is a dog or a cat or a human.

24. The pharmaceutical formulation of claim 1 wherein the omega-3 fatty acids are derived from a source selected from Norwegian salmon, Perilla frutesceus or Linum usitatissimum.
